(19) United States
(12) Patent Application Publication
(10) Pub. No.: US 2026/0253718 A1
(43) Pub. Date: Aug. 27, 2026

(54) METHOD, SYSTEM AND NON-TRANSITORY COMPUTER-READABLE RECORDING MEDIUM FOR ASSISTING ANALYSIS OF BIO-SIGNAL BY USING CLUSTERING

(71) Applicant: HUINNO, CO., LTD., Seoul (KR)

(72) Inventors: Jun Ho AN, Seoul (KR); Kwon Woo HA, Seoul (KR); Kwang Lo LEE, Hanam-si, Gyeonggi-do (KR); Sung Hoon JUNG, Seoul (KR); Jin Guk KIM, Seoul (KR); Jae Seong JANG, Seoul (KR)

(21) Appl. No.: 18/879,075

(22) PCT Filed: Aug. 14, 2023
(86) PCT No.: PCT/KR2023/012039

§ 371 Date: Jan. 23, 2025

(30) Foreign Application Priority Data
Aug. 30, 2022 (KR) 10-2022-0109506

(51) Int. Cl. G16H 40/63

(52) U.S. Cl. CPC G16H 40/63

(57) ABSTRACT

Provided according to an aspect of the present invention is a method for assisting in analysis of a bio-signal by using clustering, the method comprising the steps of: extracting features from multiple pieces of bio-signal data with respect to a bio-signal and, on the basis of the extracted features, generating multiple feature vectors for the multiple pieces of bio-signal data respectively; and performing clustering of the multiple pieces of bio-signal data with reference to the multiple feature vectors.

### CROSS-REFERENCE TO RELATED APPLICATIONS

This application is a National Stage Entry of International Application No. PCT/KR2023/012039 filed Aug. 14, 2023, which claims priority from Korean Application No. 10-2022-0109506 filed Aug. 30, 2022. The aforementioned applications are incorporated herein by reference in their entireties.

### FIELD OF THE INVENTION

The present invention relates to a method, system, and non-transitory computer-readable recording medium for assisting in biosignal analysis using clustering.

### BACKGROUND

Recently, wearable monitoring devices that can continuously monitor biosignals during daily life without hospital visits have been introduced. The biosignals measured through the wearable monitoring devices may be analyzed by automated or intelligent biosignal analysis models, and various advanced technologies are utilized to enhance the accuracy of the analysis.

Although the application of advanced technologies such as artificial neural networks to the analysis models has increased the accuracy of the analysis, a final determination or diagnosis regarding the biosignals still requires medical personnel (e.g., doctors or interpretation specialists) to inspect the analysis results of the biosignal analysis models.

However, biosignal data measured over several days or weeks (or analysis result data for the biosignal data) can be vast, amounting to hundreds of thousands of entries, which poses a problem that medical personnel spend a significant amount of time and effort inspecting all the data individually.

In particular, when the biosignals are electrocardiogram (ECG) signals, the important features of the signals (e.g., shapes of QRS complexes) may appear differently depending on the characteristics of the measuring devices or the individual subjects being measured. Therefore, even when AI-based analysis models are used, it is difficult to analyze the ECG signals with 100% accuracy solely with the analysis models, making it essential for medical personnel to personally inspect the ECG signals.

For example, assuming that an ECG signal with a normal heart rate of 60 BPM is measured for 14 days, a total of over 1.2 million pieces of beat (which is a data unit including a QRS complex) data will be generated. Even if only about 1% of that data corresponds to abnormal data, 12,000 pieces of abnormal beat data will be produced, requiring medical personnel to spend an enormous amount of effort and time identifying and inspecting such a vast amount of data individually.

In this regard, the inventor(s) have devised a technique for assisting medical personnel in efficiently inspecting a plurality of pieces of biosignal data (or their analysis results) with similar characteristics (e.g., shapes, rhythms, or patterns) by accurately and efficiently clustering the biosignal data.

### DISCLOSURE

### Technical Problem

One object of the present invention is to solve all the above-described problems.

Another object of the invention is to allow medical personnel to efficiently inspect biosignal data and its analysis results on the basis of clusters, without experiencing the inefficiency of thoroughly inspecting the biosignal data, by extracting features from a plurality of pieces of biosignal data for a biosignal, and generating a plurality of feature vectors for the plurality of pieces of biosignal data, respectively, on the basis of the extracted features, and performing clustering on the plurality of pieces of biosignal data with reference to the plurality of feature vectors.

Yet another object of the invention is to utilize various information obtained from biosignal data to improve the accuracy of clustering and adjust the number of clusters situationally, thereby enhancing both the efficiency and reliability of inspection of biosignal data analysis results by medical personnel.

### Technical Solution

The representative configurations of the invention to achieve the above objects are described below.

According to one aspect of the invention, there is provided a method for assisting in biosignal analysis using clustering, the method comprising the steps of: extracting features from a plurality of pieces of biosignal data for a biosignal, and generating a plurality of feature vectors for the plurality of pieces of biosignal data, respectively, on the basis of the extracted features; and performing clustering on the plurality of pieces of biosignal data with reference to the plurality of feature vectors.

According to another aspect of the invention, there is provided a system for assisting in biosignal analysis using clustering, the system comprising: a feature extraction unit configured to extract features from a plurality of pieces of biosignal data for a biosignal, and generate a plurality of feature vectors for the plurality of pieces of biosignal data, respectively, on the basis of the extracted features; and a clustering management unit configured to perform clustering on the plurality of pieces of biosignal data with reference to the plurality of feature vectors.

In addition, there are further provided other methods and systems to implement the invention, as well as non-transitory computer-readable recording media having stored thereon computer programs for executing the methods.

### Advantageous Effects

According to the invention, it is possible to allow medical personnel to efficiently inspect biosignal data and its analysis results on the basis of clusters, without experiencing the inefficiency of thoroughly inspecting the biosignal data.

According to the invention, it is possible to utilize various information obtained from biosignal data to improve the accuracy of clustering and adjust the number of clusters situationally, thereby enhancing both the efficiency and reliability of inspection of biosignal data analysis results by medical personnel.

### BRIEF DESCRIPTION OF THE DRAWINGS

FIG. 1 schematically shows the configuration of an entire system according to one embodiment of the invention.

FIG. 2 illustratively shows the internal configuration of a biosignal analysis system according to one embodiment of the invention.

FIG. 3 illustratively shows biosignal data to be clustered according to one embodiment of the invention.

FIG. 4 illustratively shows the structure of a model used for feature extraction according to one embodiment of the invention

FIG. 5 illustratively shows a result of performing hierarchical clustering according to one embodiment of the invention.

FIG. 6 illustratively shows a degree to which interpretation efficiency of an inspector increases as clustering is performed according to one embodiment of the invention.

FIGS. 7 to 10 illustratively show results of performing clustering on ECG signal data according to one embodiment of the invention.

### DESCRIPTION OF THE REFERENCE NUMERALS

100: communication network
200: biosignal analysis system
210: feature extraction unit
220: clustering management unit
230: communication unit
240: control unit
300: Device

### BEST MODES FOR CARRYING OUT THE INVENTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the locations or arrangements of individual elements within each of the disclosed embodiments may also be modified without departing from the spirit and scope of the invention. Therefore, the following detailed description is not to be taken in a limiting sense, and the scope of the invention, if properly described, is limited only by the appended claims together with all equivalents thereof. In the drawings, like reference numerals refer to the same or similar functions throughout the several views.

Hereinafter, preferred embodiments of the invention will be described in detail with reference to the accompanying drawings to enable those skilled in the art to easily implement the invention.

### Configuration of the Entire System

Hereinafter, a preferred embodiment of a biosignal analysis system according to the invention will be discussed in detail.

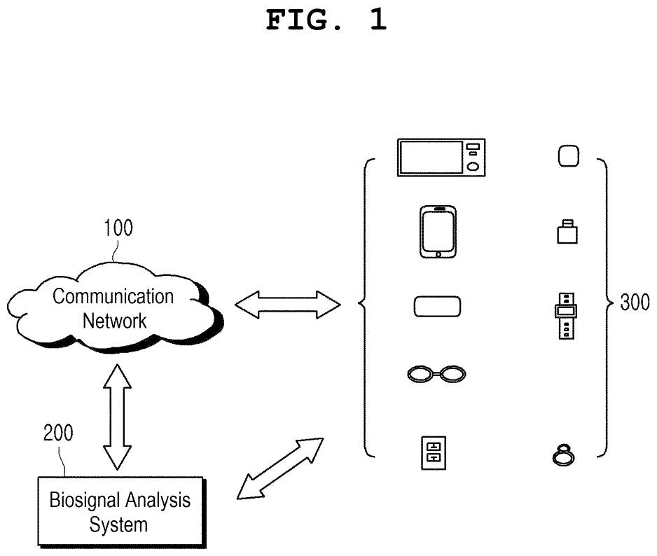

FIG. 1 schematically shows the configuration of the entire system according to one embodiment of the invention.

As shown in FIG. 1, the entire system according to one embodiment of the invention may comprise a communication network 100, a biosignal analysis system 200, and a device 300.

First, the communication network 100 according to one embodiment of the invention may be implemented regardless of communication modality such as wired and wireless communications, and may be constructed from a variety of communication networks such as local area networks (LANs), metropolitan area networks (MANS), wide area networks (WANs). Preferably, the communication network 100 described herein may include known short-range wireless communication networks such as Wi-Fi, Wi-Fi Direct, LTE Direct, and Bluetooth. However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as WiFi communication, WiFi-Direct communication, Long Term Evolution (LTE) communication, Bluetooth communication (including Bluetooth Low Energy (BLE) communication), infrared communication, and ultrasonic communication. As another example, the communication network 100 may be an optical communication network, at least a part of which may be implemented with a conventional communication scheme such as LiFi (Light Fidelity).

Next, the biosignal analysis system 200 according to one embodiment of the invention may function to extract features from a plurality of pieces of biosignal data for a biosignal, and generate a plurality of feature vectors for the plurality of pieces of biosignal data, respectively, on the basis of the extracted features, and to perform clustering on the plurality of pieces of biosignal data with reference to the plurality of feature vectors.

Meanwhile, the above description is illustrative although the biosignal analysis system 200 has been described as above, and it is apparent to those skilled in the art that at least some of the functions or components required for the biosignal analysis system 200 may be implemented or included in the device 300, as necessary.

Lastly, the device 300 according to one embodiment of the invention is digital equipment that may function to connect to and then communicate with the biosignal analysis system 200, and any type of digital equipment having a memory means and a microprocessor for computing capabilities may be adopted as the device 300 according to the invention. The device 300 may be a wearable device such as smart glasses, a smart watch, a smart patch, a smart band, a smart ring, and a smart necklace, or may be a somewhat traditional device such as a smart phone, a smart pad, a desktop computer, a notebook computer, a workstation, a personal digital assistant (PDA), a web pad, and a mobile phone.

In particular, the device 300 according to one embodiment of the invention may include a sensing means (e.g., a contact electrode or an infrared sensor) for acquiring a biosignal from a human body, and a display means for providing a user with a variety of information on the measurement of the biosignal.

In addition, according to one embodiment of the invention, the device 300 may further include an application program for performing the functions according to the invention. The application may reside in the device 300 in the form of a program module. The nature of the program module may be generally similar to those of a feature extraction unit 210, a clustering management unit 220, a communication unit 230, and a control unit 240 of the biosignal analysis system 200 to be described below. Here, at least a part of the application may be replaced with a hardware or firmware device that may perform substantially equal or equivalent functions, as necessary.

### Configuration of the Biosignal Analysis System

Hereinafter, the internal configuration of the biosignal analysis system 200 crucial for implementing the invention and the functions of the respective components thereof will be discussed.

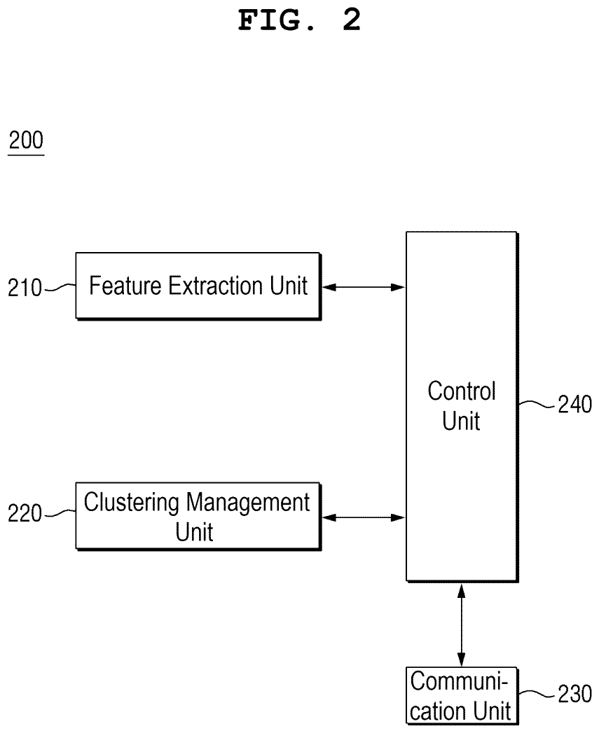

FIG. 2 illustratively shows the internal configuration of the biosignal analysis system according to one embodiment of the invention.

Referring to FIG. 2, the biosignal analysis system 200 according to one embodiment of the invention may comprise a feature extraction unit 210, a clustering management unit 220, a communication unit 230, and a control unit 240. According to one embodiment 4 the invention, at least some of the feature extraction unit 210, the clustering management unit 220, the communication unit 230, and the control unit 240 of the biosignal analysis system 200 may be program modules to communicate with an external system (not shown). The program modules may be included in the biosignal analysis system 200 in the form of operating systems, application program modules, and other program modules, while they may be physically stored in a variety of commonly known storage devices. Further, the program modules may also be stored in a remote storage device that may communicate with the biosignal analysis system 200. Meanwhile, such program modules may include, but are not limited to, routines, subroutines, programs, objects, components, data structures and the like for performing specific tasks or executing specific abstract data types as will be described below in accordance with the invention.

Meanwhile, it is noted that according to one embodiment of the invention, the biosignal analysis system 200 may operate independently without communication with the outside, in which case the communication unit 230 may not be included in the biosignal analysis system 200.

First, the feature extraction unit 210 according to one embodiment of the invention may acquire a plurality of pieces of biosignal data measured from a subject's body. Here, the biosignal data may be electrocardiogram (ECG) signal data measured from the subject's body, and the ECG signal data may be composed of beat units that include QRS complexes.

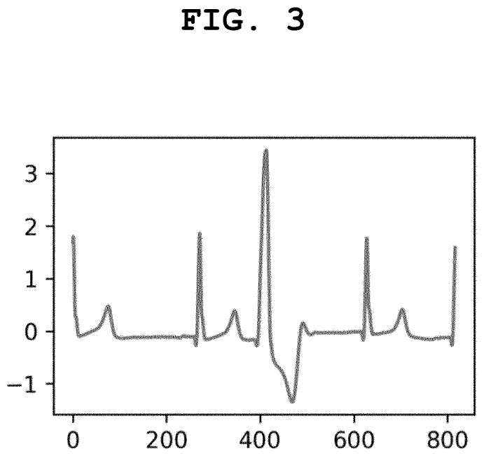

For example, the ECG signal data to be clustered may be composed of a sequence of five beats, and an interval from an R peak of the first beat to an R peak of the last beat may be defined as one piece of ECG signal data (see FIG. 3).

Further, the feature extraction unit 210 according to one embodiment of the invention may extract features from a plurality of pieces of biosignal data for a biosignal, and generate a plurality of feature vectors for the plurality of pieces of biosignal data, respectively, on the basis of the extracted features.

For example, the feature extraction unit 210 according to one embodiment of the invention may extract the features from the biometric signal data using a feature extraction model trained through supervised learning.

According to one embodiment of the invention, significant changes in signal values appear in QRS complexes included in an ECG signal, and thus when clustering is performed with respect to raw data of the biosignal data, there may occur a problem that the clustering is not properly performed due to beat position differences, beat height differences, baseline variations, noises, and the like. According to the invention, features are extracted from the raw data to generate feature vectors and clustering is performed with respect to the feature vectors, so that it is possible to reduce the problem that may occur when the clustering is performed with respect to the raw data.

Further, the feature extraction unit 210 according to one embodiment of the invention may normalize a plurality of pieces of raw data and extract features from the normalized data. This allows the length of the clustered data to be identical (or uniform) even when the length of the raw data varies due to various factors such as heart rate.

In addition, the feature extraction unit 210 according to one embodiment of the invention may extract features from a plurality of pieces of raw data using a feature extraction model, and the plurality of pieces of raw data may be normalized according to the method described above and inputted to the feature extraction model. For example, the feature extraction model may be a model obtained by optimizing a squeeze & excitation (SE) ResNet model according to the objects of the invention.

Meanwhile, the feature extraction unit 210 according to one embodiment of the invention may extract scaled R-R intervals that reflect the characteristics of rhythm between beats within a plurality of pieces of raw data for an ECG signal, and the extracted scaled R-R intervals may be used as a criterion for clustering to be described below.

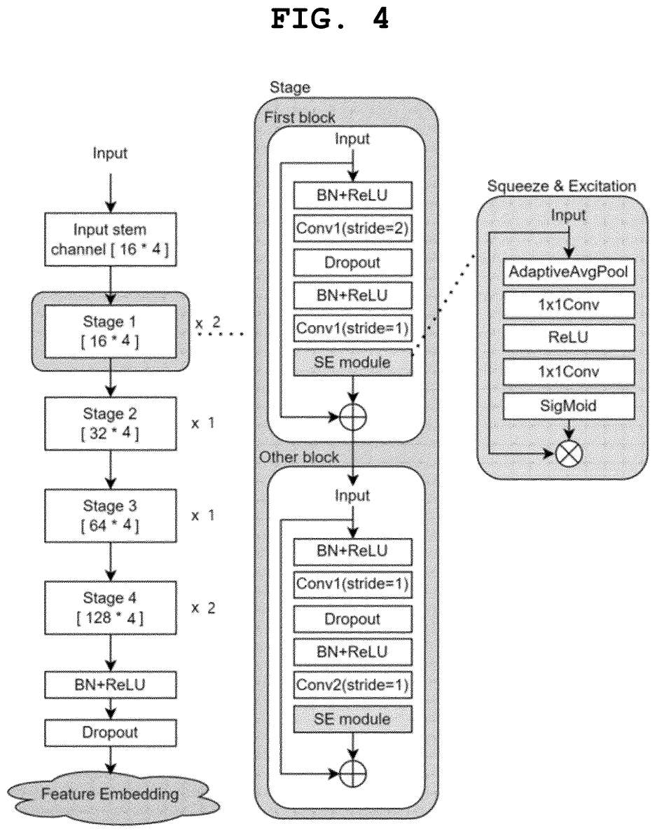

FIG. 4 illustratively shows the structure of a model used for feature extraction according to one embodiment of the invention. However, it is noted that the feature extraction unit 210 according to the invention is not necessarily implemented on the basis of the model shown in FIG. 4, but may be changed without limitation as long as the objects of the invention may be achieved.

Next, the clustering management unit 220 according to one embodiment of the invention may acquire analysis result data for a plurality of pieces of biosignal data from a biosignal analysis model. According to one embodiment of the invention, the biosignal analysis model may be a model that outputs an analysis result regarding whether the analyzed biosignal data corresponds to arrhythmia, or an analysis result regarding what type of arrhythmia the analyzed biosignal data corresponds to.

For example, according to one embodiment of the invention, the biosignal analysis model may analyze biosignal data of a subject using a machine learning algorithm (e.g., an artificial neural network) to calculate a score regarding whether the biosignal data corresponds to (or does not correspond to) a normal state in terms of arrhythmia.

As another example, according to one embodiment of the invention, the biosignal analysis model may analyze biosignal data of a subject using a machine learning algorithm (e.g., an artificial neural network) to calculate a score regarding whether the biosignal data corresponds to (or does not correspond to) a specific type of arrhythmia.

Here, according to one embodiment of the invention, the score calculated by the biosignal analysis model may encompass a value for at least one of a probability, a vector, a matrix, and a coordinate regarding correspondence (or non-correspondence) to a normal state or a specific type of arrhythmia.

Meanwhile, the biosignal that may be analyzed by the biosignal analysis model may include a signal regarding an electrocardiogram (ECG), an electromyogram (EMG), an electroencephalogram (EEG), a photoplethysmogram (PPG), a heartbeat, a body temperature, a blood sugar level, a pupil change, a blood pressure level, a blood oxygen content, and the like.

Next, the clustering management unit 220 according to one embodiment of the invention may perform clustering on a plurality of pieces of first-type biosignal data analyzed as corresponding to a first type by the biosignal analysis model, among the plurality of pieces of biosignal data.

Here, according to one embodiment of the invention, the first type refers to, in its broadest sense, a type that may be determined by the biosignal analysis model, and may encompass a normal state in terms of arrhythmia, an abnormal state in terms of arrhythmia, and a state corresponding to a specific type of arrhythmia (e. g., atrial premature complexes (APCs), ventricular premature complexes (VPCs), atrial fibrillation (AFib), and paroxysmal supra ventricular tachycardia (PSVT)).

Specifically, the clustering management unit 220 according to one embodiment of the invention may cluster a plurality of pieces of first-type biosignal data analyzed as corresponding to the first type into at least one cluster. According to one embodiment of the invention, the biosignal data clustered into the same cluster as the clustering is performed may have features (e.g., patterns, feature points, or waveforms) that are common to each other.

For example, the algorithm that may be used for the biosignal data clustering according to one embodiment of the invention may include complete linkage clustering, k-means, mean shift, Gaussian mixture model (GMM), density-based spatial clustering of applications with noise (DBSCAN), and self-organizing map (SOM). However, it is noted that the clustering algorithm according to the invention is not necessarily limited to those listed above but may be changed without limitation as long as the objects of the invention may be achieved.

In addition, the clustering management unit 220 according to one embodiment of the invention may perform clustering on the plurality of extracted feature vectors.

Further, the clustering management unit 220 according to one embodiment of the invention may perform clustering on the plurality of pieces of biosignal data corresponding to the plurality of feature vectors, with reference to the plurality of feature vectors.

For example, the biosignal data (or the plurality of pieces of raw data) according to one embodiment of the invention may include data classified as APCs or data classified as VPCs, and the clustering management unit 220 according to one embodiment of the invention may perform clustering on the data classified as APCs and clustering on the data classified as VPCs separately.

Further, the clustering management unit 220 according to one embodiment of the invention may perform clustering on the biosignal data using a hierarchical clustering technique. According to the hierarchical clustering technique utilized in the invention, there are advantages that the number of clusters does not need to be predetermined and structural relationships between the clusters may be easily identified. Furthermore, the hierarchical clustering may generate only the level (or number) of clusters suitable for analysis or interpretation situations, thereby preventing unnecessary clustering and further enhancing the efficiency of interpretation (or inspection).

Specifically, the clustering management unit 220 according to one embodiment of the invention may perform the clustering with reference to a distance map between the plurality of feature vectors generated on the basis of the features extracted from the plurality of pieces of biosignal data. For example, pieces of biosignal data corresponding to certain feature vectors may be clustered to belong to different clusters as a distance between the feature vectors is longer, and may be clustered to belong to the same cluster as the distance between the feature vectors is shorter.

More specifically, the clustering management unit 220 according to one embodiment of the invention may perform the clustering with further reference to a distance map between the plurality of pieces of raw data. For reference, the distance map between the plurality of pieces of raw data may be treated as an indicator representing differences between shapes or positions of QRS complexes included in the raw data. For example, when a distance difference between certain pieces of raw data is greater than a predetermined value (i.e., when shapes or positions of QRS complexes are different), the clustering management unit 220 according to one embodiment of the invention may impose a predetermined penalty on a distance map between feature vectors corresponding to the pieces of raw data, thereby allowing morphological similarity between pieces of biosignal data appearing in the raw data to be reflected in the clustering.

Meanwhile, the clustering management unit 220 according to one embodiment of the invention may perform complete linkage clustering with reference to the distance map calculated as above.

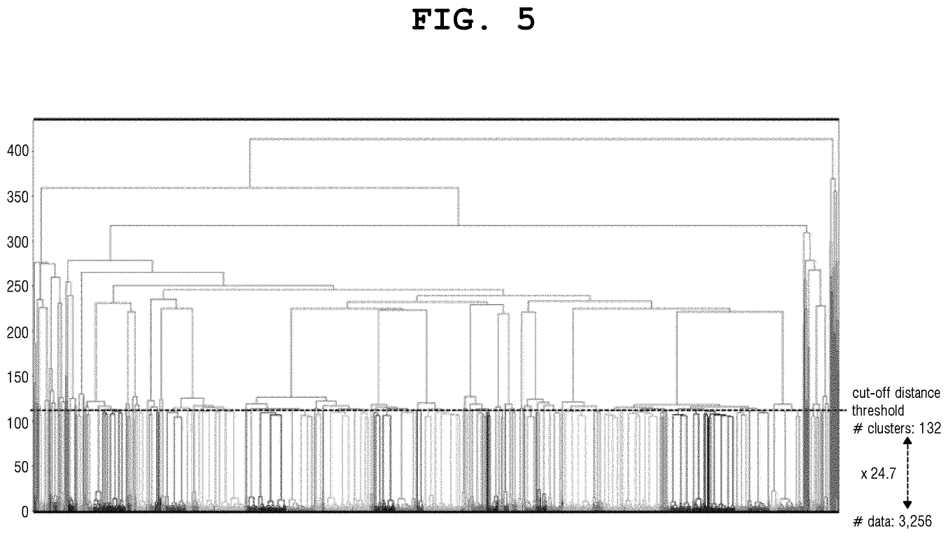

Further, the clustering management unit 220 according to one embodiment of the invention may determine the hierarchy (or number) of clusters to be outputted as a result of the clustering, on the basis of a cut-off distance threshold determined by a ratio of the number of clusters to the total number of pieces of raw data in hierarchical clusters constructed as the result of the clustering. For example, as shown in FIG. 5, when a ratio of inspection efficiency increase due to the clustering (i.e., the total number of pieces of raw data/the number of clusters) should be 20 times or more and the total number of pieces of raw data is 3,256, a hierarchy that generates a total of 132 clusters may be determined as an output hierarchy, thereby achieving an inspection efficiency increase ratio of 24.7.

That is, when a ratio of inspection efficiency increase due to the clustering is 20, a medical worker (or inspector) only needs to inspect one cluster including twenty pieces of ECG signal data instead of inspecting each of the twenty pieces of ECG signal data individually, so that the inspection efficiency may be increased by about 20 times due to the clustering according to the invention.

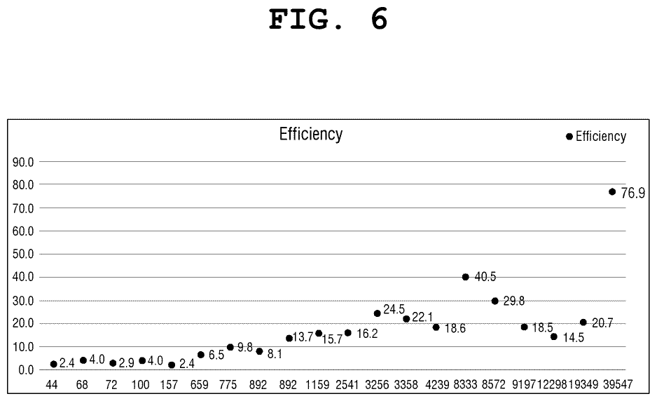

Meanwhile, FIG. 6 illustratively shows a degree to which interpretation efficiency of an inspector increases as clustering is performed according to one embodiment of the invention. Referring to FIG. 6, it can be seen that a ratio of inspection efficiency increase (shown on the vertical axis of FIG. 6) due to the clustering is greater as there are more pieces of clustered biosignal data (shown on the horizontal axis of FIG. 6).

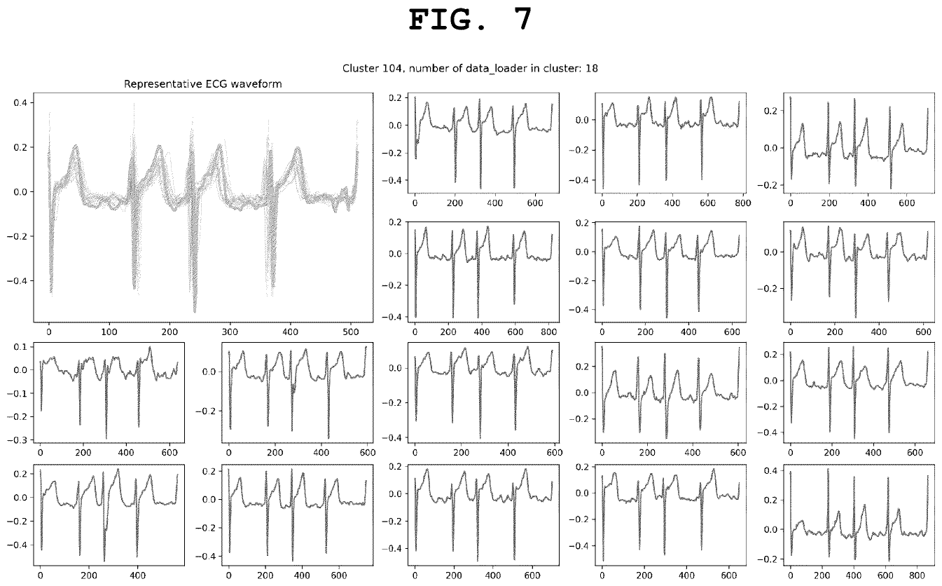

Meanwhile, FIGS. 7 to 10 illustratively show results of performing clustering on ECG signal data according to one embodiment of the invention.

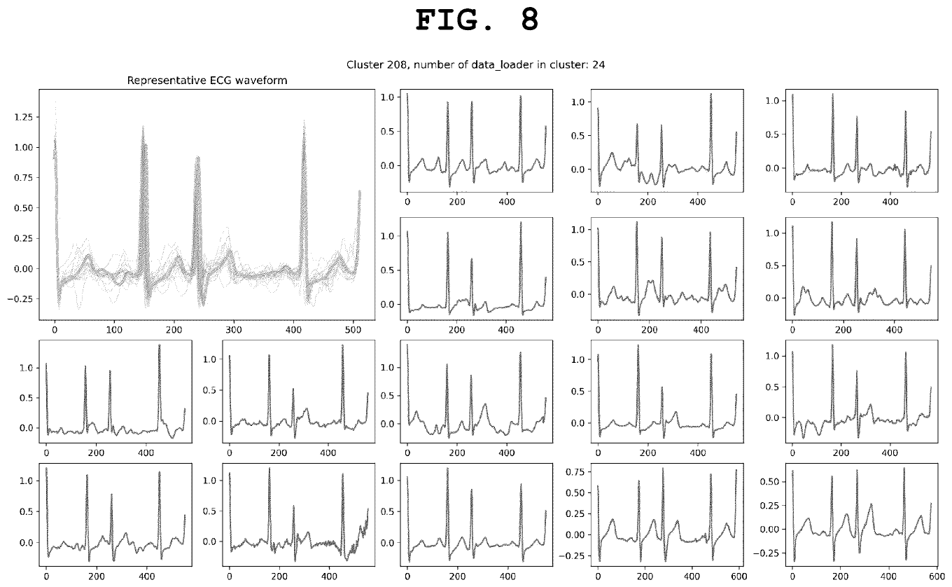

Referring to FIGS. 7 and 8, a result is shown in which 18 pieces of ECG signal data classified as APCs are clustered into one cluster (see FIG. 7), and a result is shown in which 24 pieces of ECG signal data classified as APCs are clustered into one cluster (see FIG. 8).

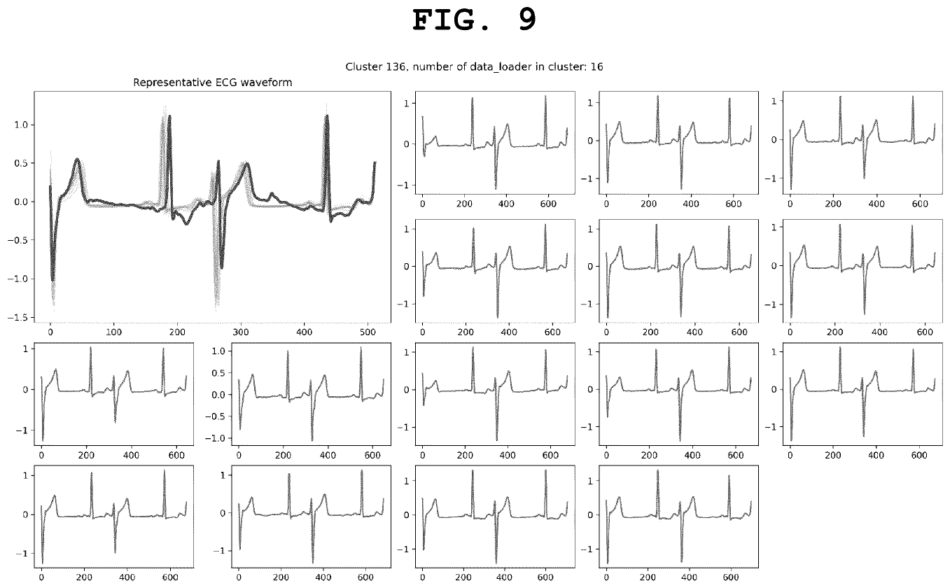

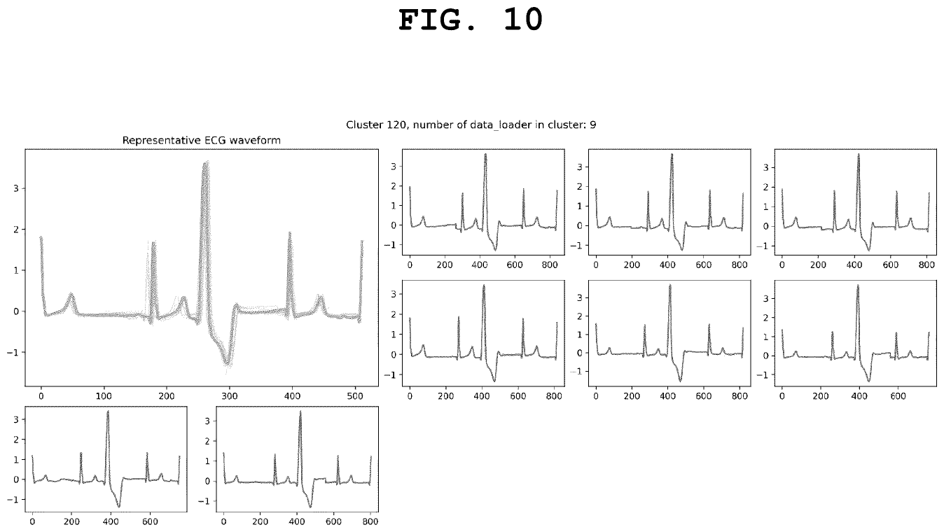

Referring to FIGS. 9 and 10, a result is shown in which 16 pieces of ECG signal data classified as VPCs are clustered into one cluster (see FIG. 9), and a result is shown in which 9 pieces of ECG signal data classified as VPCs are clustered into one cluster (see FIG. 10).

Next, the communication unit 230 according to one embodiment of the invention may function to enable data transmission/reception from/to the feature extraction unit 210 and the clustering management unit 220.

Lastly, the control unit 240 according to one embodiment of the invention may function to control data flow among 44 the feature extraction unit 210, the clustering management unit 220, and the communication unit 230. That is, the control unit 240 according to the invention may control data flow into/out of the biosignal analysis system 200 or data flow among the respective components of the biosignal analysis system 200, such that the feature extraction unit 210, the clustering management unit 220, and the communication unit 230 may carry out their particular functions, respectively.

Although the embodiments for analyzing ECG signal data have been mainly described above, it is noted that the signal that may be analyzed according to the invention is not necessarily limited only to an ECG signal, but the present invention may be utilized for other types of biosignals without limitation, as long as the objects of the invention may be achieved.

The embodiments according to the invention as described above may be implemented in the form of program instructions that can be executed by various computer components, and may be stored on a non-transitory computer-readable recording medium. The non-transitory computer-readable recording medium may include program instructions, data files, data structures and the like, separately or in combination. The program instructions stored on the non-transitory computer-readable recording medium may be specially designed and configured for the present invention, or may also be known and available to those skilled in the computer software field. Examples of the non-transitory computer-readable recording medium include the following: magnetic media such as hard disks, floppy disks and magnetic tapes; optical media such as compact disk-read only memory (CD-ROM) and digital versatile disks (DVDs) ; magneto-optical media such as floptical disks; and hardware devices such as read-only memory (ROM), random access memory (RAM) and flash memory, which are specially configured to store and execute program instructions. Examples of the program instructions include not only machine language codes created by a compiler or the like, but also high-level language codes that can be executed by a computer using an interpreter or the like. The above hardware devices may be configured to operate as one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

1. A method for assisting in biosignal analysis using clustering, the method comprising the steps of:
extracting features from a plurality of pieces of biosignal data for a biosignal, and generating a plurality of feature vectors for the plurality of pieces of biosignal data, respectively, on the basis of the extracted features; and
performing clustering on the plurality of pieces of biosignal data with reference to the plurality of feature vectors.

2. The method of claim 1, wherein in the step of extracting the features, the plurality of pieces of biosignal data are normalized and the features are extracted from the plurality of pieces of normalized biosignal data.

3. The method of claim 1, wherein in the step of performing the clustering, clustering on a plurality of pieces of biosignal data for a biosignal corresponding to a first classification is performed separately from clustering on a plurality of pieces of biosignal data for a biosignal corresponding to a second classification.

4. The method of claim 1, wherein in the step of performing the clustering, the clustering on the plurality of pieces of biosignal data is performed with reference to a distance map between the feature vectors.

5. The method of claim 4, wherein in the step of performing the clustering, the clustering on the plurality of pieces of biosignal data is performed with further reference to a distance map between raw data of the plurality of pieces of biosignal data.

6. The method of claim 1, wherein in the step of performing the clustering, hierarchical clustering is performed on the plurality of pieces of biosignal data, and a number of clusters to be outputted as a result of the clustering is determined on the basis of inspection efficiency for the biosignal data.

7. A non-transitory computer-readable recording medium having stored thereon a computer program for executing the method of claim 1.

8. A system for assisting in biosignal analysis using clustering, the system comprising:
a feature extraction unit configured to extract features from a plurality of pieces of biosignal data for a biosignal, and generate a plurality of feature vectors for the plurality of pieces of biosignal data, respectively, on the basis of the extracted features; and
a clustering management unit configured to perform clustering on the plurality of pieces of biosignal data with reference to the plurality of feature vectors.

9. The system of claim 8, wherein the feature extraction unit is configured to normalize the plurality of pieces of biosignal data and extract the features from the plurality of pieces of normalized biosignal data.

10. The system of claim 8, wherein the clustering management unit is configured to perform clustering on a plurality of pieces of biosignal data for a biosignal corresponding to a first classification separately from clustering on a plurality of pieces of biosignal data for a biosignal corresponding to a second classification.

11. The system of claim 8, wherein the clustering management unit is configured to perform the clustering on the plurality of pieces of biosignal data with reference to a distance map between the feature vectors.

12. The system of claim 11, wherein the clustering management unit is configured to perform the clustering on the plurality of pieces of biosignal data with further reference to a distance map between raw data of the plurality of pieces of biosignal data.

13. The system of claim 8, wherein the clustering management unit is configured to perform hierarchical clustering on the plurality of pieces of biosignal data, and determine a number of clusters to be outputted as a result of the clustering on the basis of inspection efficiency for the biosignal data.
